(19) United States
(12) Patent Application Publication
(10) Pub. No.: US 2022/0192557 A1
(43) Pub. Date: Jun. 23, 2022

(54) WEB-BASED VIRTUAL LIGHTBAR AND METHOD OF PROVIDING EMDR THERAPY ONLINE IN RELIANCE UPON THE SAME

(71) Applicants: Amie Luyties, Long Beach, CA (US); Michael Luyties, Long Beach, CA (US)

(72) Inventors: Amie Luyties, Long Beach, CA (US); Michael Luyties, Long Beach, CA (US)

(21) Appl. No.: 17/130,144

(22) Filed: Dec. 22, 2020

(51) Int. Cl. A61B 5/16; A61B 5/00; G16H 20/70; G16H 40/67

(52) U.S. Cl. CPC A61B 5/165; A61B 5/163; G16H 40/67; G16H 20/70; A61B 5/742

(57) ABSTRACT

A system for providing psychotherapy is disclosed, including a server configured to transmit information via a network to a client device and a therapist device. An application program is in operable communication with the server. The application program operates an EMDR therapy including bilateral stimulation therapy, wherein the bilateral stimulation therapy is comprised of a lightbar continuously moving across an interactive display screen of the client device and the therapist device. The lightbar's movement is controlled by the therapist utilizing a lightbar controller provided on the interactive display screen of the therapist device. The therapy may also include Brainspotting therapy wherein the lightbar is moved along the x-axis and the y-axis to identify problem points.

### CROSS-REFERENCE TO RELATED APPLICATIONS

The present application claims priority to U.S. Provisional Patent Application 63/011,401 filed on Apr. 17, 2020, entitled “WEB-BASED VIRTUAL LIGHTBAR AND METHOD OF PROVIDING EMDR THERAPY ONLINE IN RELIANCE UPON THE SAME” the entire disclosure of which is incorporated by reference herein.

### TECHNICAL FIELD

The embodiments generally relate to eye movement desensitization and reprocessing (EMDR) treatment and, more particularly, relate to a web-based EMDR lightbar and audio bilateral stimulator to effect audio bilateral stimulation (BLS) and a method of providing EMDR therapy or treatment online via the Internet or world-wide web, in reliance upon the web-based lightbar and audio bilateral stimulator.

### BACKGROUND

Eye Movement Desensitization and Reprocessing (EMDR) is a method of psychological therapy for the treatment of a range of psychological disorders and mental health problems including trauma, anxiety, Post-traumatic Stress Disorder (PTSD), among others. During EMDR therapy, the therapist aids the client in processing distressing experiences and guides the client through a series of bilateral stimulations (BLS's), involving side-to-side sensory stimulation. Often, a light is moved back and forth across a screen, a sound is switched between speakers on either side of the client, a vibration is moved back and forth between vibrating devices, or a combination thereof. Brainspotting is a similar technique to EMDR which also includes vertical stimulation and lateral stimulation.

Remote psychotherapy has grown in practice in recent years; however, EMDR and Brainspotting are interactive forms of therapy between the therapist and the client which can be difficult to replicate remotely. Many systems in the current arts allow the client to control the stimulation while the therapist interacts with the client.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments provided herein relate to a system for providing psychotherapy, including a server configured to transmit information via a network to a client device and a therapist device. An application program is in operable communication with the server. The application program operates an EMDR therapy, including bilateral stimulation therapy, wherein the bilateral stimulation therapy is comprised of a lightbar continuously moving across an interactive display screen of the client device and the therapist device. The lightbar's movement is controlled by the therapist utilizing a lightbar controller provided on the interactive display screen of the therapist device. The therapy may also include Brainspotting therapy wherein the lightbar is moved along the x-axis and the y-axis to identify problem points.

The embodiments provide a web-based EMDR lightbar and audio bilateral stimulator to effect audio stimulation (BLS). The web-based EMDR lightbar and audio bilateral stimulator are preferably implemented by computer code, i.e., an application program, programmed to do so and be operational in a server. The server, as used herein, is a remote computer that receives data from the therapist and sends the data to the client in real-time, ensuring that the therapist is always in control of the therapy session, via the Internet. The web-based EMDR lightbar and audio bilateral stimulator are preferably coordinated, i.e., the sounds produced by the audio bilateral stimulator “follow” the path and perturbations of the EMDR lightbar.

In one aspect, the embodiments provide a method of providing EMDR therapy or treatment online, via the Internet or world-wide web, in reliance upon the web-based lightbar and audio bilateral stimulator. The method is implemented at a computer server in response to commands or/data communicated by a therapist to the application program operating on the server. The application program (server) establishes a HIPAA compliant connect that is secured with end-to-end encryption, with the client receiving the therapy. The web-based EMDR lightbar and BLS are presented to the client's electronic device such as a cell phone, tablet, laptop computer, desktop computer, etc. (without limitation), while the therapist and client communicate by phone and or an audio connection implemented by the method.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1A illustrates a system flow diagram highlighting the process of implementing a web-based audio and video therapy session, which sends the web-based EMDR lightbar and audio bilateral stimulation to the client's electronic device as therapeutic communications are exchanged between the therapist and client, according to some embodiments;

FIG. 1B illustrates a flow diagram of a method implemented by a set of computer readable instructions operated upon by a microprocessor at the server, according to some embodiments;

FIG. 2 illustrates a display screen presented to the therapist wherein the therapist enters their information including their name and login credentials, according to some embodiments;

FIG. 3 illustrates a display screen including a start button that is selected on by the therapist to start a session and to highlight control of the light color and sound of the inventive web-based EMDR lightbar and audio bilateral stimulator, according to some embodiments;

FIG. 4 illustrates the light generated by the web-based lightbar that is presented in real-time to the client's display device, in a first lightbar position, wherein the lightbar moves continuously in real-time from the left edge of the screen, synchronously with sounds output from a sound producing device comprising at least two output ports, such as an audio headset or a pair of loudspeakers, according to some embodiments;

FIG. 5 illustrates the light generated by the web-based light bar moved to the right (relative to the position in FIG. 4) in the user or clients display device, according to some embodiments;

FIG. 6 illustrates the light generated by the web-based light bar moved to the right (relative to the position shown in FIG. 5) in the user or clients display device, according to some embodiments;

FIG. 7 illustrates the light generated by the web-based light bar moved to the right (relative to the position shown in FIG. 6) in the user or clients display device, according to some embodiments;

FIG. 8 illustrates the light generated by the web-based light bar moved to the right (relative to the position shown in FIG. 7) in the user or clients display device, according to some embodiments;

FIG. 9 illustrates the light generated by the web-based light bar moved to the left (relative to the position shown in FIG. 8) in the user or clients display device, according to some embodiments;

FIG. 10 illustrates the light generated by the web-based light bar moved to the left (relative to the position shown in FIG. 9) in the user or clients display device, according to some embodiments;

FIG. 11 illustrates the interactive display screen including a stop button that may be selected by the therapist to end the therapy session, according to some embodiments;

FIG. 12 illustrates an interactive display screen of the brainspotting interface to provide remote brainspotting therapy, wherein the lightbar is moving along the x-axis, according to some embodiments;

FIG. 13 illustrates an interactive display screen of the brainspotting interface to provide remote brainspotting therapy wherein the lightbar is moving along the y-axis, according to some embodiments; and

FIG. 14 illustrates an interactive display screen of the brainspotting interface to provide remote brainspotting therapy, wherein the lightbar is moving along the x-axis and y-axis, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to systems and methods for providing an online EMDR-based therapy session that relies upon the web-based EMDR lightbar and audio bilateral stimulator online, via the Internet or world-wide web.

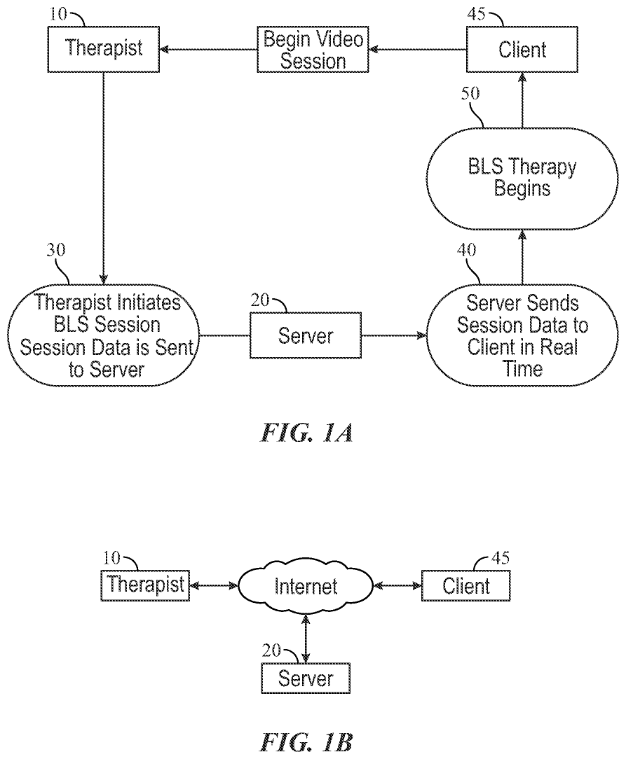

FIG. 1A illustrates a system flow diagram highlighting the process of implementing a web-based audio and video therapy session, including a therapist device (i.e., a therapist computer-based electronic device) 10. The therapist uses his/her device 10 to access the application program operational at the server 20, which is operational at a website, to initiate a BLS session 30. Consequently, the application program operates via the server 20 to send session data to the client (i.e., the client's computer-based electronic device) 45 via the Internet 40, wherein the actual BLS session begins 50. Preferably, the application program operating at the website via the server includes an application programming interface (API) that presents the interactive display screens in response to the therapist/client inputs. FIG. 1B depicts an embodiment wherein the method is implemented by a set of computer readable instructions operated upon by a microprocessor at the server to implement the steps of the method. The therapist (i.e., therapist device) 10 interacts with the client (i.e., client device) 45 via a network (e.g., the Internet). Server 20 operates the application program to provide the functionalities described herein.

Processors suitable for the execution of a computer program include both general and special purpose microprocessors and any one or more processors of any digital computing device. The processor will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system and other devices attached to a network, such as other computer systems, or between nodes of the computer system. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

The steps and actions of the computer system described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor such that the processor can read information from, and write information to, the storage medium. In the alternative, the storage medium may be integrated into the processor. Further, in some embodiments, the processor and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

The method of providing EMDR and/or BLS online therapy sessions in reliance upon the lightbar and bilateral stimulator is implemented at a computer server in response to commands or data communicated by a therapist to the application program operating on the server. The application program, via the server, establishes a HIPAA compliant connection that is secured with end-to-end encryption, with the client receiving the therapy.

As used herein, the terms “client” and “user” describe a person receiving the BLS. The term “therapist” is used herein to describe the person initiating the BLS therapy session. The phrase “video session” is used herein to describe a standard video conference between the therapist and client and can utilize a video service to create a secure link between the therapist and the client for the application program operating at the server 20. As used herein, the phrase bilateral stimulation (or bilateral stimulator; “BLS”) means lateral stimulation implemented by light or sound, that when presented to a user, stimulates both sides of the user's brain periodically, according to the frequency of the signal. For example, the BLS may be visual BLS, wherein a light goes from one side of the screen to another left, right, left, right, and so forth positions.

The sound plays in synchronization to the moving light on the left side of the headset (i.e., to the left ear) for 1.5 second or less (for example, 1 second) when the light reaches the left edge of the screen. The sound plays on the right side of the headset (i.e., to the right ear) for 1.5 seconds or less (for example, 1 second) when the light reaches the right edge of the screen. While use of a headset as a sound producing device is preferred, the client 45 can use any pair of sound generating devices, such as conventional loudspeakers, as long as the client's electronic device can synchronize with and drive the loudspeakers so that the first of the pair of loudspeakers outputs the sound as the light is in the left half of the display screen and second pair of loudspeakers outputs the sound when the light is in the right half of the display screen.

The therapist 10 controls the speed, size, and color of the light from their computer, tablet, phone, or like computing device, in cooperation with the inventive process (implemented as the application program at server 20). The therapist 10 also controls when the light starts and stops from their computing device, as should be clear to persons having ordinary skill in the art when considering the entirety of the disclosures herein. As the therapist 10 clicks start/stop or changes the speed or dimensions of the light, the client receives the light on their computer, tablet, phone, or like computing device, and sees the light start/stop and anything else the therapist has initiated for their viewing.

The phrase “audio BLS” as used herein means a sound/tone which goes from one side of the headset (plugged into the client's device) to the opposing side, e.g., left ear, right ear, left ear, right ear, and so forth. Preferably, the sound is synchronized to the moving light in the displayed images at the client's display device during the therapy session. In one implementation, “session data,” which comprises the data that is sent from the therapist 20 to the client 45, includes:
1. Start/Stop;
2. Audio Sound Effect;
   a. Tone; and
   b. Volume;
3. Lightbar Settings;
4. Speed;
5. Size;
6. Color;
7. Shape; and
8. Luminance.

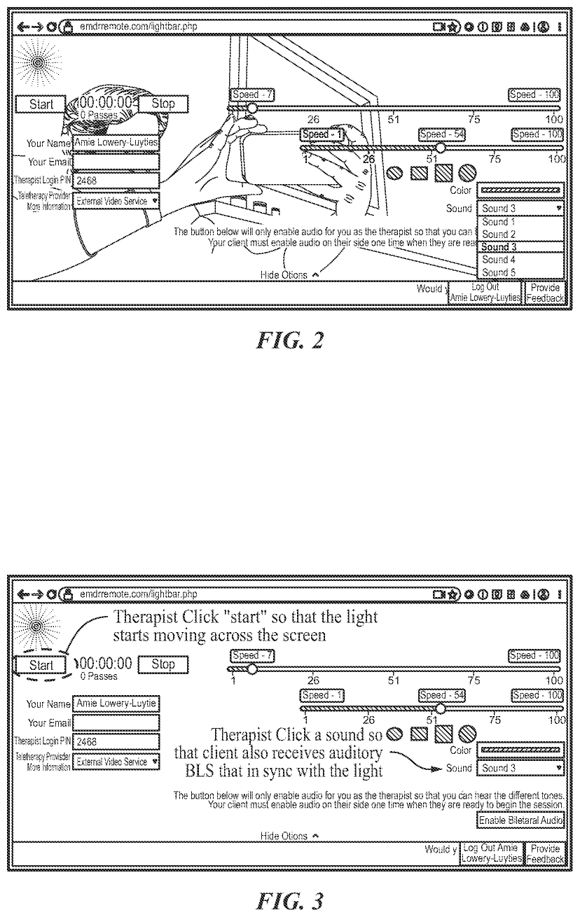

As known to one skilled in the arts, the application program comprises a set of computer readable instructions stored in a memory (in the server 20), accessible by a processor (in the server 20), which are operated upon by the processor to implement the embodiments. The application program or its API, when accessed by a therapist 10 or client 45 device, orchestrates the embodiments, highlighted by the display images presented in FIGS. 2-14.

FIG. 2 illustrates an exemplary interactive display screen presented to a user, whether the user is a therapist or a client, by the application program, which allows a user to join a therapy session as a therapist or client, wherein the display screen includes a button to be pressed if the user is a therapist. In such, the therapist may be on their electronic device (e.g., a desktop computer), which is in a different location than the client. The therapist clicks on the address bar on any browser window and enters the associated webpage whereon the therapist logs into their account by selecting the therapist button. Selecting the therapist button transfers the user to a therapist login screen illustrated in FIG. 3.

FIG. 3 illustrates an interactive display screen presented to the therapist once the therapist has accessed the website using the interactive display screen illustrated in FIG. 2. The therapist types their name in the search box and enters their associated password.

During initiation of the system, the interactive display screen provided an interactive display wherein the users and therapists login to the system using login credentials. The interactive display screen including a button to be pressed if the user is a client. The client is utilizing an electronic device which is at a remote location than the therapist. The client then selects a button to transfer the client to the client login screen.

The interactive display screen is presented to the client after the client has accessed the website using the interactive display screen depicted (client login), to identify his/her therapist. The client types their therapists name in the search box. The interactive display screen is presented to the client after processing the therapist information input by the client using the interactive display screen, where the session with the therapist will be initiated if the client agrees to the terms of service. After the client types in the therapist name, a box will appear for the client to write their name (first name only) and the client will click a box to “agree to terms” of using the online therapy tool. Then the client will automatically be signed into the therapists virtual waiting room.

The interactive display screen is presented to the therapist identified by the client in the interactive screen. Once the client signs into the waiting room, the therapist will be alerted on their screen (on the left side of the screen). The alert on the therapist screen will show the name that the client typed in. The therapist sees the client's name displayed in the virtual waiting and the therapist clicks on the client's name so they can join together in the session remotely (they are both at different location and are connecting remotely online).

FIG. 2 is an exemplary interactive display screen presented to the therapist to gather therapist-related information and to allow the therapist to control the therapy session. The therapist may change light and audio settings remotely (i.e., from their computer device as connected and logged into the server or application program) that will appear on the client's display screen and in the client's audio device, for example, speakers or headphones attached to the client's electronic computer device. On this screen, the therapist can change the size, shape, and speed of the light moving across the screen, which is in sync with the audio tone moving from one side of the client's headset to the other. When the therapist changes the settings, the light on the client's screen changes size, shape, and/or speed, depending on what the therapist sets the controls at. The therapist can change the type of sound/tone and the speed of the tone, which will be in sync with the light (e.g., when the light is on the left, the tone will be on the left).

FIG. 3 illustrates an exemplary interactive display screen of FIG. 2, including the “start” button that is clicked on by the therapist to start a session, and to highlight control of the light color and sound of the inventive web-based EMDR lightbar and audio bilateral stimulator, respectively. Once the controls are set by the therapist, the therapist then clicks the rectangular “start” button located on the top left side of the screen. The client cannot see the control panel or the start/stop button. The client will only see the light and, if the audio is enabled, the client will also hear the audio tone in their headset/headphone. The light will appear on the client's screen while the client is in a different location from the therapist, and the audio will make sounds on the client's headset in sync with the light. The functions are controlled by the therapist remotely through the operation of the application program at the server.

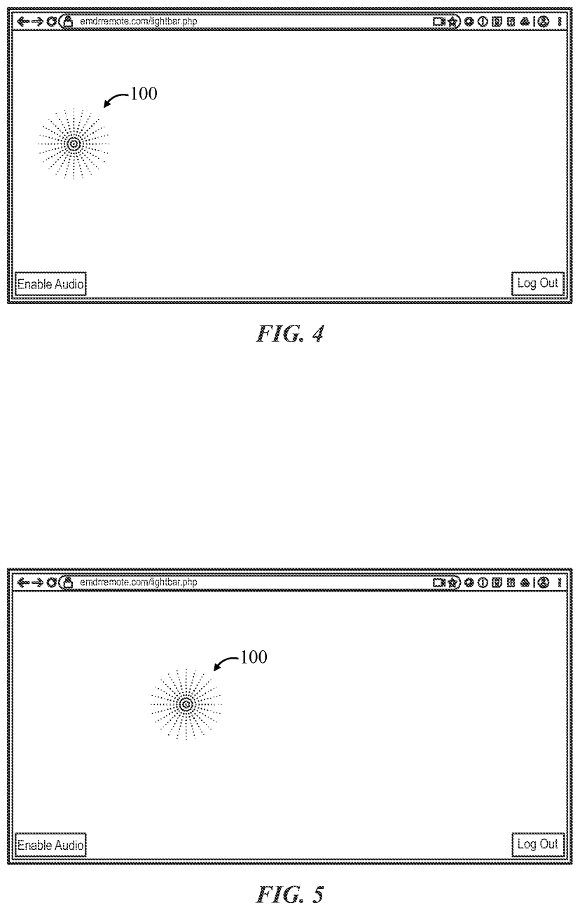

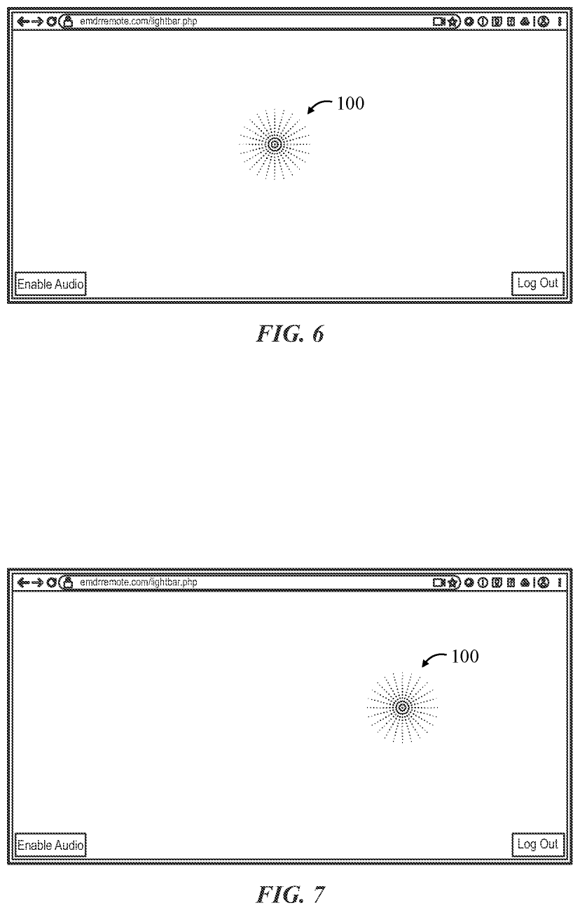

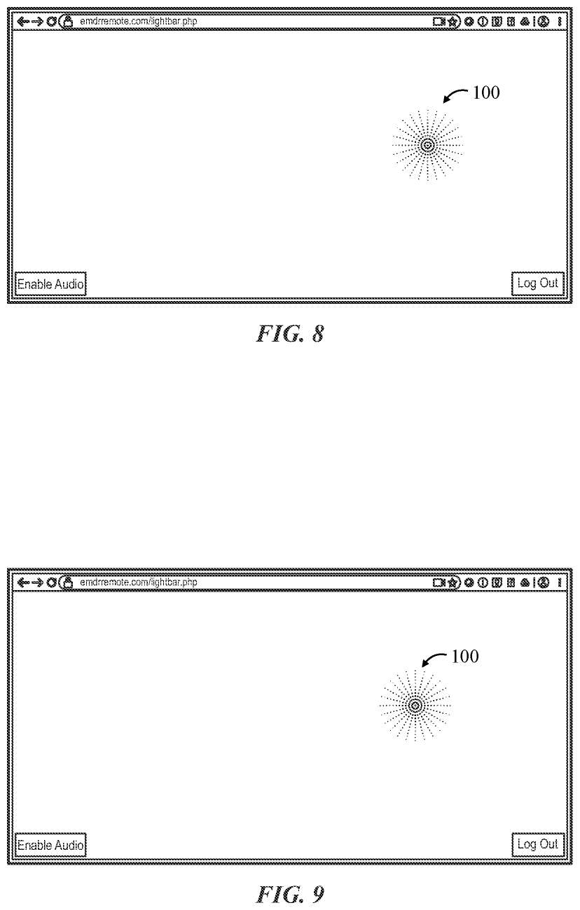

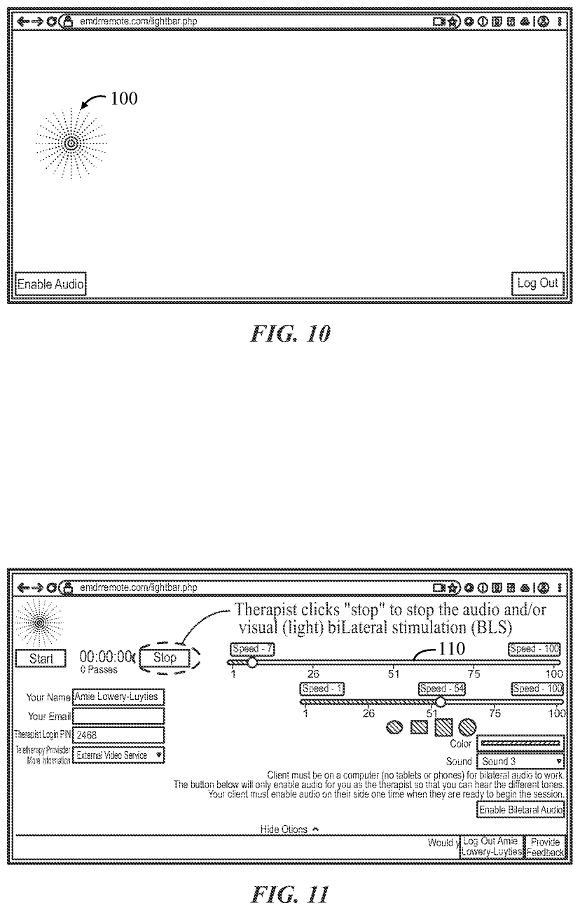

FIGS. 4-10 illustrate the lightbar 100 operation, i.e., the bilateral stimulation. FIG. 4 presents a displayed image comprising the light generated by the web-based lightbar 100 as shown in real-time on the client's display device according to the embodiments provided herein, in a first light bar position. FIG. 5 illustrates the lightbar 100 in a second position wherein the lightbar has advanced to the right, relative to FIG. 4. FIG. 6 illustrates the lightbar 100 in a third position wherein the lightbar 100 has advanced to the right, relative to FIG. 5. FIG. 7 illustrates the lightbar 100 in a fourth position wherein the lightbar 100 has advanced to the right, relative to FIG. 6. FIG. 8 illustrates the lightbar 100 in a fifth position wherein the lightbar has advanced to the right, relative to FIG. 7, before changing lateral direction to the left as depicted by the light's position advance to the left in FIG. 9, relative to its position in FIG. 8, wherein the lightbar is illustrated in a sixth position. FIG. 10 illustrates the lightbar in a seventh position wherein the lightbar has advanced to the left, relative to its position in FIG. 9.

While the images of the lightbar presented in FIGS. 4-10 are still images, the lightbar 100 moves continuously in real-time during the operation of the system.

In greater detail, the client, on his/her computing device connected to the server, sees the light move from left to right across the screen, which is being controlled by the therapist remotely. In such, the therapist is using their computer, tablet, phone, or like computing device at a different location than the client, who is on their computer, tablet, phone, or like computing device. The lightbar 100 begins moving continuously from left to right (FIGS. 4-8) and then right to left (FIGS. 9-10). If the audio is enabled (e.g., by pressing an “enable audio” button presented to the therapist at the bottom of the screen depicted on FIG. 11), then the audio will also start on the client's computer and will sync with the light, such that the light and audio tone/sound will be in sync and controlled by the therapist remotely. For example, as the light is at the left of the screen, the audio tone will be presented on the left side of the client (i.e., to the left ear); when the light is on the right of the screen, the audio tone will be presented on the right side of the client (i.e., to the right ear).

In FIGS. 4-10, the lightbar 100 moves linearly, by all appearances in real-time (e.g., the images in the figures depict a display device that was refreshed every 1/60^{th} of a second), from the left edge of the screen to the right edge of the screen and back to the left edge of the screen again, in a continuous manner, synchronously with the sound data. While still images of the lightbar 100 position are provided, the lightbar 100 will move continuously when operating the system. The client's EMDR or BLS therapy may be said to be optimized by use of the methods, as the light and sound presented at the user's electronic device display device and sound producing device affect two of the client senses, synchronously. That is, the client is bilaterally stimulated through sensory input captured by his/her eyes and bilaterally stimulated by sensory input captured by his/her ears, synchronously.

FIG. 4 illustrates the lightbar 100 (and/or audio) on the client's computer display device all the way on the left side of the screen as controlled remotely by the therapist. If the left side of the screen represents zero and the right side represents 100, then the lightbar is currently at 0 (in FIG. 4). FIG. 5 shows the light (and/or audio) advanced 25% across the screen, as the method is controlled or configured remotely by the therapist, wherein if the left side of the screen represents zero and the right side represents 100, then the light and audio of FIG. 5 is currently at 25. Similarly, FIG. 6 depicts the light further advanced to the right another 25% of the width of the display screen to the 50% point. If the left side of the screen represents zero and the right side represents 100, then the light and audio of FIG. 6 is currently at 50.

FIG. 7 depicts the lightbar 100 (and/or audio) on the client's computer display device after having moved about 75% across the screen as controlled remotely by the therapist. If the left side of the screen represents zero and the right side represents 100, then the light and audio of FIG. 7 is currently at 75, and wherein FIG. 8 further shows the light advanced the entire width of the display device screen (i.e., completely on the right side of the screen). If the left side of the screen represents zero and the right side represents 100, then the light (and/or) audio of FIG. 8 is currently at 100.

From the right-most screen position depicted in FIG. 8, the light begins moving laterally left, as shown in FIG. 9. FIG. 10 depicts the lightbar 100 (and/or audio) on the client's computer display device having been moved back to the position shown in FIG. 4. This process of the light and/or audio goes back and forth (i.e., repeating the cycle illustrated in FIGS. 4-10) with continuous movement across the screen until the therapist decides the treatment is over.

While the figures may suggest that the lightbar 100 moves discontinuously at 25% increments, the lightbar 100 in fact moves continuously across the display. FIGS. 4-10 illustrate the moments wherein the lightbar 100 has moved in 25% increments of the display screen, in each “next” position. The lightbar 100, in the eyes of the client viewing the lightbar 100 on his/her display device, moves continuously right to left, left to right, again right to left, and so on. The embodiments provide that a “next image” is displayed at a refresh-rate between 1/25 and 1/75 of a second, preferably 1/60 of a second. As long as the client's electronic device can process the incoming lightbar 100 and any sound data synchronously therewith to affect a display rate of at least 1/25 second, which most display devices known in the arts are capable of, then any such known display device capable of employing the system described herein may be used by the client, whereby the synchronized light and sound are sensed by the client continuously.

FIG. 11 is a version of the exemplary interactive display screen of FIG. 2, including the “stop” button that is clicked on by the therapist to end the therapy session. The therapist stops the BLS (light and/or audio) remotely from their computing device in communication with the server, and the light and/or audio stops on the client's computer once the therapist has pressed “stop” on the displayed image presented by the application program via the server on the display screen of the therapist's computer or other electronic device. A lightbar controller 110 allows the therapist to control the movement of the lightbar 100 across the interactive display screen as illustrated in FIGS. 4-10. The lightbar controller 110 allows the therapist to control the speed, intensity, color, shape, size, etc. of the lightbar 100 during the therapy session.

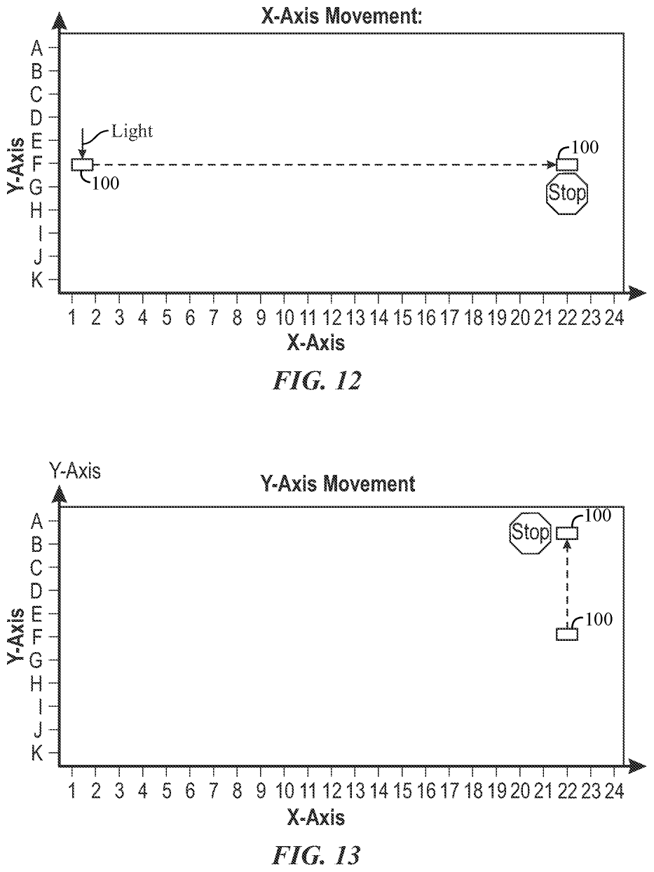

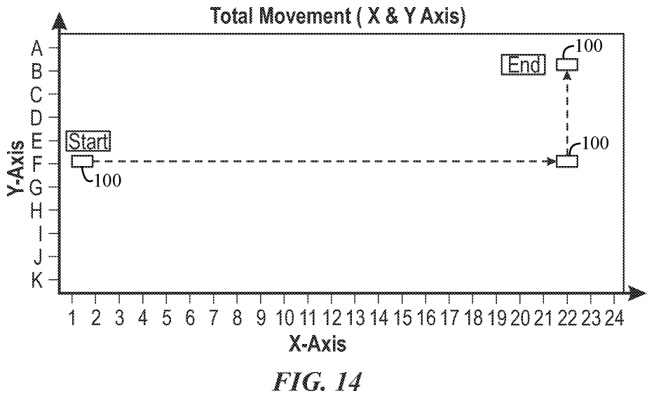

FIGS. 12-14 relate to a system for providing remote brainspotting therapy in an embodiment of the present invention. Similar to the EMDR therapy illustrated in FIGS. 4-10 hereinabove, the therapist controls the lightbar's 100 movement, which may include light and/or sound stimulation, along the x-axis (left to right and/or right to left). Once a “problem point” is identified on the x-axis, the therapist stops the movement along the x-axis and changes the movement direction to move the lightbar 100 along the y-axis (up to down and/or down to up). For example, the lightbar 100 may start at the left side of the display of the client's computing device and move along the x-axis toward the right side of the display while staying at a midpoint on the y-axis. Once the problem point along the x-axis is established, the therapist moves the lightbar 100 up and/or down as shown in FIG. 13 and FIG. 14. This process is repeated until one or more problem points are established (e.g., between 1 and 6 problem points). The therapist may then record the locations of the problem points (e.g., with x- and y-axis coordinates) to display the problem points to the client to show one or more lightbars 100 each corresponding to a problem point. For example, the therapist may alter the lightbar 100 presentation throughout the therapy session accordingly to address each of the identified problem points.

As used herein, the term “problem point” may relate to a lightbar 100 location on the display during EMDR therapy, bilateral stimulation, and/or Brainingspotting wherein the client experiences an emotion (e.g., discomfort, anxiety, etc.) or other experience. The problem point can be saved or otherwise noted by the therapist and/or client and revisited later in the therapy session or future therapy session. The display may be configured to depict multiple problem points on the same interactive display interface. interface.

In specific reference to FIG. 12, during Brainspotting the lightbar 100 is moved by the therapist horizontally along the x-axis until a problem point is reached. The problem point is identified by the therapist and/or the client, and the lightbar's 100 movement along the x-axis is stopped. The therapist then moves the lightbar 100 up or down along the y-axis to identify another problem point as shown in FIG. 13 and FIG. 14.

In some embodiments, the memory stores client data including the x- and y-axis coordinates of problem points. The memory may also store client data input by the client and/or the therapist related to therapy session information, client/therapist interactions during or outside of the therapy session, client reactions to problem points and experiences throughout the therapy session, and the like.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A system for providing psychotherapy, the system comprising:
a server configured to transmit information via a network to a client device and a therapist device; and
an application program in operable communication with the server, the application program to operate an EMDR therapy including bilateral stimulation therapy, wherein the bilateral stimulation therapy is comprised of a lightbar continuously moving across an interactive display screen of the client device and the therapist device, and wherein the lightbar's movement is controlled by the therapist utilizing a lightbar controller provided on the interactive display screen of the therapist device.

2. The system of claim 1, further comprising a memory to store client data.

3. The system of claim 2, wherein the client data includes client experiences throughout a therapy session.

4. The system of claim 1, wherein the client data is comprised of one or more problem points identified by the client or the therapist.

5. The system of claim 1, wherein the client and therapist connect to a website managed by the server via the Internet.

6. The system of claim 1, wherein the step of using a computer server includes managing and preparing a database of therapists.

7. The system of claim 1, further comprising the step of securely maintaining communications between a client and a therapist.

8. The system of claim 4, further comprising the step of securely maintaining client data.

9. A system for providing psychotherapy, the system comprising:
a server configured to transmit information via a network to a client device and a therapist device;
an application program in operable communication with the server, the application program to operate an EMDR therapy including bilateral stimulation therapy, comprised of a lightbar continuously moving across an interactive display screen of the client device and the therapist device, and wherein the lightbar's movement is controlled by the therapist utilizing a lightbar controller provided on the interactive display screen of the therapist device; and
wherein the application program is in operable communication with the server to operate a Brainspotting therapy wherein the lightbar moves laterally along the x-axis and vertically along the y-axis, wherein the lightbar's movement is controlled by the therapist utilizing the lightbar controller.

10. The system of claim 9, wherein the lightbar position is controlled by the therapist and moves on the interactive display screen on the client's device in real-time.

11. The system of claim 9, further comprising a memory to store client data.

12. The system of claim 11, wherein the memory is configured as a HIPAA compliant database.

13. The system of claim 12, wherein the client data includes client experiences throughout a therapy session.

14. The system of claim 13, wherein the client data is comprised of one or more problem points identified by the client or the therapist.

15. The system of claim 14, wherein the client and therapist connect to a website managed by the server via the Internet.

16. The system of claim 15, wherein the step of using a computer server includes managing and preparing a database of therapists.

17. The system of claim 16, further comprising the step of securely maintaining communications between a client and a therapist.

18. The system of claim 17, further comprising the step of securely maintaining client data.

19. The system of claim 18, wherein the therapist may display each problem point and alter the lightbar setting at each problem point.

20. A method for providing psychotherapy, the method comprising the steps of:
transmitting information via a server in operable communication with a network to a client device and a therapist device, the information associated with an application program in operable communication with the server;
operating, via the application program, an EMDR therapy including bilateral stimulation therapy and Brainspotting therapy;
moving, via the therapist using an interactive display screen, a lightbar, wherein the lightbar is continuously moving across the interactive display screen on the client's device; and
identifying, via the client or the therapist, one or more problem point locations an x-axis and a y-axis.
